(19) United States
(12) Patent Application Publication
(10) Pub. No.: US 2026/0125440 A1
(43) Pub. Date: May 7, 2026

(54) INCRETIN ANALOGS AND USES THEREOF

(71) Applicant: ELI LILLY AND COMPANY, Indianapolis, IN (US)

(72) Inventors: Milata Mary ABRAHAM, Indianapolis, IN (US); Jorge ALSINA-FERNANDEZ, Indianapolis, IN (US); Tamer COSKUN, Carmel, IN (US); Hongchang QU, Carmel, IN (US); James Lincoln WALLIS, Indianapolis, IN (US)

(21) Appl. No.: 19/298,995

(22) Filed: Aug. 13, 2025

Related U.S. Application Data
(63) Continuation of application No. 17/785,758, now Pat. No. 12,410,227.
(60) Provisional application No. 62/949,661, filed on Dec. 18, 2019.

(51) Int. Cl. C07K 14/575; A61K 38/00; A61P 3/04; A61P 3/06; A61P 3/10

(52) U.S. Cl. CPC C07K 14/575; A61P 3/04; A61P 3/06; A61P 3/10; A61K 38/00

(57) ABSTRACT

Incretin analogs are provided that have activity at each of the glucose-dependent insulinotropic polypeptide (GIP), glucagon-like peptide-1 (GLP-1) and glucagon (GCG) receptors. The incretin analogs have structural features resulting in balanced activity and extended duration of action at each of these receptors. Methods also are provided for treating diseases such as type 2 diabetes mellitus, dyslipidemia, metabolic syndrome, non-alcoholic fatty liver disease, non-alcoholic steatohepatitis and obesity.

The present application is being filed along with a Sequence Listing in ST.26 XML format. The Sequence Listing is provided as a file titled “X₂₂₆₂₂A_US_000 SequenceListing” created Aug. 13, 2025, and is 39,402 bytes in size. The Sequence Listing information in the ST.26 XML format is incorporated herein by reference in its entirety. No new matter is added herewith.

The disclosure relates to incretin analogs having activity at each of a glucose-dependent insulinotropic polypeptide (GIP), glucagon-like peptide-1 (GLP-1) and glucagon (GCG) receptors. The incretin analogs herein have structural features that provide balanced activity at each of these receptors and that have an extended duration of action. Such incretin analogs may be useful for treating conditions, diseases and disorders including type 2 diabetes mellitus (T2DM), dyslipidemia, metabolic syndrome, non-alcoholic fatty liver disease (NAFLD), non-alcoholic steatohepatitis (NASH) and/or obesity, as well as cardiovascular diseases that are not considered metabolic diseases and neurodegenerative diseases.

Over the past several decades, the prevalence of diabetes continues to rise. T2DM is the most common form of diabetes accounting for about 90% of all diabetes. T2DM is characterized by high blood glucose levels stemming from insulin resistance. The current standard of care for T2DM includes diet and exercise, as well as treatment with oral medications and injectable glucose-lowering drugs including incretin-based therapies, such as GLP-1 receptor agonists.

GLP-1 is a 36-amino acid peptide, the major biologically active fragment of which is produced as a 30-amino acid, C-terminal amidated peptide (GLP-17-36; SEQ ID NO:2) that stimulates glucose-dependent insulin secretion and that prevents hyperglycemia in diabetics. A variety of GLP-1 analogs currently are available for treating T2DM, including dulaglutide, exenatide and liraglutide. Many currently marketed GLP-1 analogs, however, are dose-limited by gastrointestinal side effects, such as nausea and vomiting. When treatment with oral medications and incretin-based therapies is insufficient, treatment with insulin is considered. Despite the treatment options available, significant numbers of individuals receiving approved therapies are not reaching glycemic control goals (see, e.g., Casagrande et al. (2013) Diabetes Care 36:2271-2279).

Uncontrolled diabetes can lead to one or more conditions that impact morbidity and mortality of such individuals. One of the main risk factors for T2DM is obesity, and many individuals with T2DM (˜90%) are overweight or obese. It is documented that a decrease in body adiposity will lead to improvement in obesity-associated co-morbidities including hyperglycemia and cardiovascular events. Therefore, therapies effective in glucose control and weight reduction are needed for better disease management.

In view thereof, new therapies being studied include compounds having not only activity at a GLP-1 receptor but also activity at one or more other receptors, such as the GIP and/or GCG receptors. In fact, certain compounds already have been described as having triple receptor agonist activity (i.e., activity at each of the GIP, GLP-1 and GCG receptors). For example, Intl. Patent Application Publication No. WO 2015/067716 describes GCG analogs having triple receptor agonist activity. Similarly, Intl. Patent Application Publication No. WO 2016/198624 describes exendin-4 analogs having triple receptor agonist activity. Likewise, Intl. Patent Application Publication Nos. WO 2014/049610 and WO 2017/116204 each describe a variety of compounds having triple receptor agonist activity. Moreover, Intl. Patent Application Publication No. WO 2017/153575 describes GCG and GLP-1 co-agonists that also are stated to have GIP receptor agonist activity.

Although typically used for treating T2DM, incretins and analogs thereof having activity at one or more of the GIP, GLP-1 and/or GCG receptors also have been described as having a potential for therapeutic value in a number of other conditions, diseases or disorders, including, for example, Alzheimer's disease, bone-related disorders, dyslipidemia, metabolic syndrome, NAFLD and NASH, obesity and Parkinson's disease. See, e.g., Jall et al. (2017) Mol. Metab. 6:440-446; Carbone et al. (2016) J. Gastroenterol. Hepatol. 31:23-31; Finan et al. (2016) Trends Mol. Med. 22:359-376; Choi et al. (2017) Potent body weight loss and efficacy in a NASH animal model by a novel long-acting GLP-1/Glucagon/GIP triple-agonist (HM15211), ADA Poster 1139-P; Ding (2008) J. Bone Miner. Res. 23:536-543; Tai et al. (2018) Brain Res. 1678:64-74; Müller et al. (2017) Physiol. Rev. 97:721-766; Finan et al. (2013) Sci. Transl. Med. 5:209; Hölscher (2014) Biochem. Soc. Trans. 42:593-600.

Nevertheless, a need remains for treatments for such conditions, diseases and disorders, especially T2DM, that are capable of providing effective glucose control, with weight loss benefits and/or a favorable side effect profile. There also is a need for therapeutic agents available for use with sufficiently extended duration of action to allow for dosing as infrequently as once-a-day, thrice-weekly, twice-weekly or once a week.

The incretin analogs herein seek to meet the needs above. Accordingly, the disclosure describes incretin analogs with activity at each of the GIP, GLP-1 and GCG receptors. Advantageously, the incretin analogs herein have balanced activity at these receptors, thereby allowing for administration of doses that provide sufficient activity at each receptor to provide the benefits of agonism of that receptor while avoiding unwanted side effects associated with too much activity. Moreover, the incretin analogs herein have an extended duration of action at each of the GIP, GLP-1 and GCG receptors allowing for dosing as infrequently as once-a-day, thrice-weekly, twice-weekly or once-a-week. In this manner, the incretin analogs result in enhanced glucose control, metabolic benefits such as lowering body weight and/or improving body composition, lipid benefits such as lowering proprotein convertase subtilisin/kexin type 9 (PCSK9), and/or other benefits such as increasing bone mass or bone formation or decreasing bone resorption. The disclosure also describes effective treatments for conditions, diseases and disorders including T2DM, dyslipidemia, metabolic syndrome, NAFLD, NASH and/or obesity.

Accordingly, the disclosure first describes incretin analogs that include a base amino acid sequence of:

YX₂QGTX₆TSDX₁₀SIX₁₃LDX₁₆X₁₇AQX₂₀X₂₁FIX₂₄X₂₅LLEGGPSSGEPP
PX₃₉,

where X₂ is Aib, X₆ is αMeF(2F), X₁₀ is Y or 4Pal, X₁₃ is L or αMeL, X₁₆ is O, X₁₇ is any amino acid with a functional group available for conjugation and the functional group is conjugated to a C₁₆-C₂₂ fatty acid moiety, X₂₀ is 4Pal, Iva or αMeL, X₂₁ is A or Aib, X₂₄ is E or e, X₂₅ is Y or αMeY, and X₃₉ is E or S (SEQ ID NO:5), and where a carboxy-terminal (C-terminal) amino acid optionally is amidated, or a pharmaceutically acceptable salt thereof.

In some instances, the amino acid with the functional group available for conjugation at position X₁₇ is C, D, E, K or Q. In certain instances, the amino acid with the functional group available for conjugation at position X₁₇ is K.

In certain instances, X₂ is Aib, X₆ is αMeF(2F), X₁₀ is Y, X₁₃ is L, X₁₆ is O, X₁₇ is K, X₂₀ is 4Pal, X₂₁ is A, X₂₄ is E, X₂₅ is Y and X₃₉ is E, such that the incretin analog includes a base amino acid sequence of:

(SEQ ID NO: 6)
Y-Aib-QGT-αMeF(2F)-TSDYSILLDOKAQ-4Pal-
AFIEYLLEGGPSSGEPPPE.

In certain instances, X₂ is Aib, X₆ is αMeF(2F), X₁₀ is Y, X₁₃ is αMeL, X₁₆ is O, X₁₇ is K, X₂₀ is Iva, X₂₁ is A, X₂₄ is E, X₂₅ is αMeY and X₃₉ is S, such that the incretin analog includes a base amino acid sequence of:

(SEQ ID NO: 7)
Y-Aib-QGT-αMeF(2F)-TSDYSI-αMeL-LDOKAQ-Iva-AFIE-
αMeY-LLEGGPSSGEPPPS.

In certain instances, X₂ is Aib, X₆ is αMeF(2F), X₁₀ is 4Pal, X₁₃ is αMeL, X₁₆ is O, X₁₇ is K, X₂₀ is αMeL, X₂₁ is Aib, X₂₄ is e, X₂₅ is αMeY and X₃₉ is S, such that the incretin analog includes a base amino acid sequence of:

(SEQ ID NO: 8)
Y-Aib-QGT-αMeF(2F)-TSD-4Pal-SI-αMeL-LDOKAQ-αMeL-
Aib-FIe-αMeY-LLEGGPSSGEPPPS.

In some instances, the C₁₆-C₂₂ fatty acid moiety is conjugated to the amino acid with the functional group available for conjugation via a linker. In certain instances, the C₁₆-C₂₂ fatty acid moiety has a structure of —CO—(CH₂)ₐ—CO₂H, where a is an integer between 16 to 22. In certain instances, the fatty acid moiety is a C₁₈ diacid or a C₂₀. Likewise, and in some instances, the linker is one or more units of [2-(2-amino-ethoxy)-ethoxy)]-acetyl (AEEA), aminohexanoic acid (Ahx), E, γ-glutamic acid (γE) or combinations thereof.

In particular instances, the incretin analog herein, including the linker and the fatty acid moiety, can be one of the following:

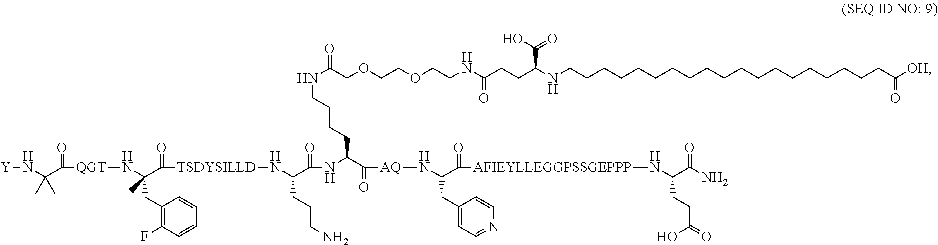

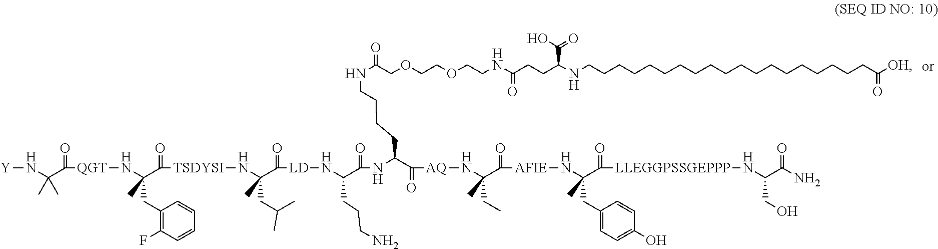

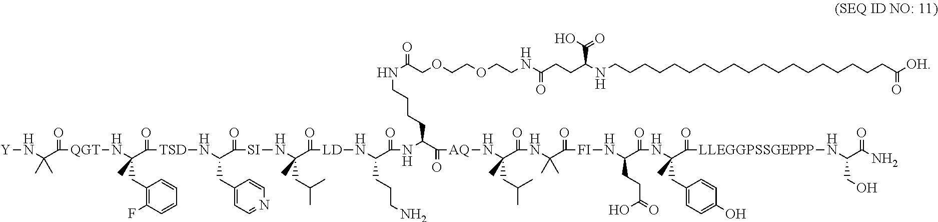

Second, pharmaceutical compositions are described that include at least one incretin analog herein or a pharmaceutically acceptable salt thereof (e.g., trifluroacetate salts, acetate salts or hydrochloride salts) and a pharmaceutically acceptable carrier. In some instances, the pharmaceutical compositions further can include carriers, diluents and/or excipients.

Third, methods are described for treating a disease such as T2DM, dyslipidemia, metabolic syndrome, NAFLD, NASH and obesity. Such methods can include at least a step of administering to an individual in need thereof an effective amount of an incretin analog described herein. In some instances, the disease is T2DM, NAFLD, NASH or obesity. In other instances, the disease is a cardiovascular disease that is not considered a metabolic disease or a neurodegenerative disease.

In some instances, the at least one incretin analog herein can be subcutaneously (SQ) administered to the individual. Likewise, and in some instances, the at least one incretin analog can be administered daily, every other day, three times a week, two times a week, one time a week (i.e., weekly), biweekly (i.e., every other week) or monthly. In certain instances, the at least one incretin analog can be administered SQ every other day, SQ three times a week, SQ two times a week, SQ one time a week, SQ every other week, or SQ once a month. In particular instances, the at least one incretin analog is administered SQ one time a week (QW).

The methods also may include steps such as measuring or obtaining the individual's weight or body composition and/or blood glucose and/or blood insulin and/or hemoglobin A1c (HbA1c) and/or blood lipid, and then comparing such obtained values to one or more baseline values or previously obtained values to assess the effectiveness of treatment.

In some instances, the individual is obese or overweight. In other instances, the individual is a person with diabetes (PwD), especially T2DM. In certain instances, the individual is obese with T2DM or overweight with T2DM.

The methods also may be combined with diet and exercise and/or may be combined with additional therapeutic agents.

Fourth, incretin analogs are described for use in therapy, such as for use in treating a disease such as T2DM, dyslipidemia, metabolic syndrome, NAFLD, NASH and obesity.

In some instances, the disease is T2DM, NAFLD, NASH or obesity. In other instances, the disease is a cardiovascular disease that is not considered a metabolic disease or a neurodegenerative disease.

Fifth, uses are described for the incretin analogs herein in manufacturing a medicament for treating T2DM, dyslipidemia, metabolic syndrome, NAFLD, NASH and obesity. In some instances, the disease is T2DM, NAFLD, NASH or obesity. In other instances, the disease is a cardiovascular disease that is not considered a metabolic disease or a neurodegenerative disease.

Unless defined otherwise, all technical and scientific terms used herein have the same meaning as commonly understood by one of skill in the art to which the disclosure pertains. Although any methods and materials similar to or equivalent to those described herein can be used in the practice or testing of the incretin analogs, pharmaceutical compositions and methods, the preferred methods and materials are described herein.

Moreover, reference to an element by the indefinite article “a” or “an” does not exclude the possibility that more than one element is present, unless the context clearly requires that there be one and only one element. The indefinite article “a” or “an” thus usually means “at least one.”

### Definitions

As used herein, “about” means within a statistically meaningful range of a value or values such as, for example, a stated concentration, length, molecular weight, pH, sequence identity, time frame, temperature or volume. Such a value or range can be within an order of magnitude typically within 20%, more typically within 10%, and even more typically within 5% of a given value or range. The allowable variation encompassed by “about” will depend upon the particular system under study, and can be readily appreciated by one of skill in the art.

As used herein, and in reference to one or more of the GIP, GLP-1 or GCG receptors, “activity,” “activate,” “activating” and the like means a capacity of a compound, such as the incretin analogs herein, to bind to and induce a response at the receptor(s), as measured using assays known in the art, such as the in vitro assays described below.

As used herein, “amino acid” means a molecule that, from a chemical standpoint, is characterized by containing one or more amine groups and one or more carboxylic acid groups, and may contain other functional groups. As is known in the art, there is a set of twenty amino acids that are designated as standard amino acids and that are used as building blocks for most of the peptides/polypeptides/proteins produced by any living being.

As used herein, “amino acid with a functional group available for conjugation” means any standard or non-standard amino acid with a functional group that may be conjugated to a fatty acid moiety by way of, for example, a linker. Examples of such functional groups include, but are not limited to, alkynyl, alkenyl, amino, azido, bromo, carboxyl, chloro, iodo and thiol groups. Additionally, examples of standard amino acids including such functional groups include C (thiol), D (carboxyl), E (carboxyl), K (amino) and Q (amide).

As used herein, “analog” means a compound, such as a synthetic peptide or polypeptide, that activates a target receptor and that elicits at least one in vivo or in vitro effect elicited by a native receptor agonist.

As used herein, “cardiovascular disease that is not considered a metabolic disease” and the like means a disease such as, for example, atherosclerosis or heart failure.

As used herein, “C₁₆-C₂₂ fatty acid” means a carboxylic acid having between 16 and 22 carbon atoms. The C₁₆-C₂₂ fatty acid suitable for use herein can be a saturated monoacid or a saturated diacid (“diacids” have a carboxyl group on each end).

As used herein, “effective amount” means an amount, concentration or dose of one or more of the incretin analogs herein, or a pharmaceutically acceptable salt thereof which, upon single or multiple dose administration to an individual in need thereof, provides a desired effect in such an individual under diagnosis or treatment (i.e., may produce a clinically measurable difference in a condition of the individual such as, for example, a reduction in blood glucose, a reduction in HbA1c, a reduction in weight or body fat and/or a change body composition). An effective amount can be readily determined by one of skill in the art by using known techniques and by observing results obtained under analogous circumstances. In determining the effective amount for an individual, a number of factors are considered, including, but not limited to, the species of mammal, its size, age and general health, the specific disease or disorder involved, the degree of or involvement or the severity of the disease or disorder, the response of the individual, the particular incretin analog administered, the mode of administration, the bioavailability characteristics of the preparation administered, the dose regimen selected, the use of concomitant medication, and other relevant circumstances.

As used herein, “extended duration of action” means that binding affinity and activity of an incretin analog continues for a period of time greater than native human GIP, GLP-1 and GCG, allowing for dosing at least as infrequently as once daily or even thrice-weekly, twice-weekly or once-weekly. The time action profile of the incretin analog may be measured using known pharmacokinetic test methods such as those utilized in the examples below.

As used herein, “glucose-dependent insulinotropic polypeptide” or “GIP” means a 42-amino acid peptide (SEQ ID NO:1) that is an incretin, which plays a physiological role in glucose homeostasis by stimulating insulin secretion from pancreatic beta cells in the presence of glucose.

As used herein, “glucagon-like peptide-1” or “GLP-1” means a 36-amino acid peptide (SEQ ID NO:2) that also is an incretin, which stimulates glucose-dependent insulin secretion and which has been shown to prevent hyperglycemia in diabetics.

As used herein, “glucagon” or “GCG” means a 29-amino acid peptide (SEQ ID NO:3) that helps maintain blood glucose by binding to and activating GCG receptors on hepatocytes, causing the liver to release glucose—stored in the form of glycogen—through a process called glycogenolysis.

As used herein, “oxyntomodulin” or “OXM” means a 37-amino acid peptide (SEQ ID NO:4) that includes not only the 29-amino acid sequence of GCG but also an octapeptide carboxy terminal extension that activates both the GCG and GLP-1 receptors, with a slightly higher potency for the GCG receptor over the GLP-1 receptor.

As used herein, “half-maximal effective concentration” or “EC₅₀” means a concentration of compound that results in 50% activation/stimulation of an assay endpoint, such as a dose-response curve (e.g., cAMP).

As used herein, “incretin analog” means a compound having structural similarities with, but multiple differences from, each of GIP, GLP-1 and GCG, especially human GIP (SEQ ID NO:1), human GLP-1 (SEQ ID NO:2) and human GCG (SEQ ID NO:3). The incretin analogs herein include amino acid sequences resulting in the compounds having affinity for and activity at each of the GIP, GLP-1 and GCG receptors (i.e., triple receptor agonist activity).

As used herein, “individual in need thereof” means a mammal, such as a human, with a condition, disease, disorder or symptom requiring treatment or therapy, including for example, those listed herein. In particular, the preferred individual to be treated is a human.

As used herein, “long-acting” means that binding affinity and activity of an incretin analog herein continues for a period of time greater than a reference peptide such as native, human GIP (SEQ ID NO:1), human GLP-1 (SEQ ID NO:2) and/or human GCG (SEQ ID NO:3), allowing for dosing at least as infrequently as once-daily, thrice-weekly, twice-weekly, once-weekly or even monthly. The time action profile of the incretin analogs herein may be measured using known pharmacokinetic methods such as those described in the Examples below.

As used herein, “non-standard amino acid” means an amino acid that may occur naturally in cells but does not participate in peptide synthesis. Non-standard amino acids can be constituents of a peptide and can be generated by modifying standard amino acids in the peptide (i.e., via post-translational modification). Non-standard amino acids can include D-amino acids, which have an opposite absolute chirality of the standard, L-amino acids above.

As used herein, “saturated” means that a fatty acid moiety that contains no carbon-carbon double or triple bonds.

As used herein, “treat,” “treating,” “to treat” and the like mean restraining, slowing, stopping or reversing the progression or severity of an existing condition, disease, disorder or symptom.

As used herein, and with reference to an incretin analog, “triple receptor agonist activity” means an incretin analog with activity at each of the GIP, GLP-1 and GCG receptors, especially an analog having a balanced and sufficient activity at each receptor to provide the benefits of agonism of that receptor while avoiding unwanted side effects associated with too much activity of that receptor. Moreover, the incretin analogs having triple receptor agonist activity have extended duration of action at each of the GIP, GLP-1 and GCG receptors, which advantageously allows for dosing as infrequently as once-a-day, thrice-weekly, twice-weekly or once-a-week.

Certain abbreviations are defined as follows: “4Pal” refers to 3-(4-pyridyl)-L-alanine; “ACR” refers to urine albumin/urine creatinine ratio; “Aib” refers to α-aminoisobutyric acid; “αMeL” refers to a-methyl leucine; “αMeK” refers to a-methyl lysine; “αMeF” refers to a-methyl phenylalanine; “αMeF(2F)” refers to α-methyl 2-fluoropheynylalanine; “αMeY” refers to a-methyl tyrosine; “amu” refers to atomic mass unit; “AUC” refers to area under the curve; “Boc” refers to tert-butoxycarbonyl; “cAMP” refers to cyclic adenosine monophosphate; “DMSO” refers to dimethyl sulfoxide; “EIA/RIA” refers to enzyme immunoassay/radioimmunoassay; “Fmoc” refers to fluorenylmethyloxycarbonyl; “hr” refers to hour or hours; “HTRF” refers to homogenous time-resolved fluorescent; “IV” refers to intravenous; “Iva” refers to isovaline; “kDa” refers to kilodaltons; “LC-MS” refers to liquid chromatography-mass spectrometry; “min” refers to minute or minutes; “MS” refers to mass spectrometry; “Om” or “0” refers to ornithine; “OtBu” refers to O-tert-butyl; “Pbf” refers to NG-2,2,4,6,7-pentamethyldihydrobenzofuran-5-sulfonyl; “RP-HPLC” refers to reversed-phase high performance liquid chromatography; “sec” refers to second or seconds; “SEM” refers to standard error of the mean; “SPA” refers to scintillation proximity assay; “SQ” refers to subcutaneous; “TFA” refers to trifluoroacetic acid; “tBu” refers to tert-butyl; and “Trt” refers to Trityl.

### Incretin Analogs

The structural features of the incretin analogs herein result in compounds having sufficient activity at each of the GIP, GLP-1 and GCG receptors to obtain the favorable effects of activity at each receptor (i.e., triple receptor agonist activity), but not so much activity at any one receptor to either overwhelm the activity at either of the other two receptors or result in undesirable side effects when administered at a dose sufficient to result in activity at all three receptors. Non-limiting examples of such structural features in certain embodiments, and with reference to SEQ ID NO:5, include αMeF(2F) at position 6, which contributes to optimal GCG, GIP and GLP-1 activity; Y or 4Pal at position 10, which contributes to balanced activity; L or αMeL at position 13, which contributes to optimal GCG and GIP activity; O at position 16, which contributes to balanced activity; acylation at position 17, which contributes to balanced activity; 4Pal, Iva or αMeL at position 20, which contributes to optimal GCG activity; A or Aib at position 21, which contributes to optimal GCG activity; Y or αMeY at position 25, which contributes to balanced activity. Other examples of such structural features include the amino acids described herein at positions 28-39, which contribute to optimal binding and potency at all three receptors.

The structural features of the incretin analogs herein also result in analogs having many other beneficial attributes relevant to their developability as therapeutic treatments, including improving the solubility of the analogs in aqueous solutions, improving chemical and physical formulation stability, extending the pharmacokinetic profile, and minimizing potential for immunogenicity. Non-limiting examples of particular structural features that result in such attributes include acylation at position 17 with a C₂₀ fatty acid, which contributes to optimal pharmacokinetic (PK) profiles and developability; 4Pal at position 10 or 20, which contributes to optimal solubility and chemical stability; O at position 16, which contributes to optimal solubility and PK; E or e at position 24, which contributes to optimal solubility and stability; Y or αMeY at position 25, which contributes to optimal chemical stability; and E or S at position 39, which contributes to optimal solubility; and the amino acids described herein at positions 6, 13, 16 and 28-39, which contribute to optimal PK, immunogenicity, developability and stability.

It should be noted that the foregoing lists of structural features are exemplary, and not comprehensive, and that the combination of beneficial characteristics of exemplary analogs described herein is not the result of any modification in isolation, but is instead achieved through the novel combinations of the structural features described herein. In addition, the above-described effects of the foregoing lists of modifications are not exclusive, as many of these modifications also have other effects important to the characteristics of the compounds described herein, as described below.

The amino acid sequences of incretin analogs herein incorporate naturally occurring (standard) amino acids, typically depicted herein using one letter codes (e.g., L=leucine; K=lysine), as well as non-standard and/or a-methyl substituted residues of standard amino acids (e.g., αMeL, αMeK, αMeF, αMeF(2F) and αMeY, and certain other non-standard amino acids, such as 4Pal, Aib, Iva, O and D-glutamic acid (e)). For clarity, the structures of these other amino acids are depicted below:

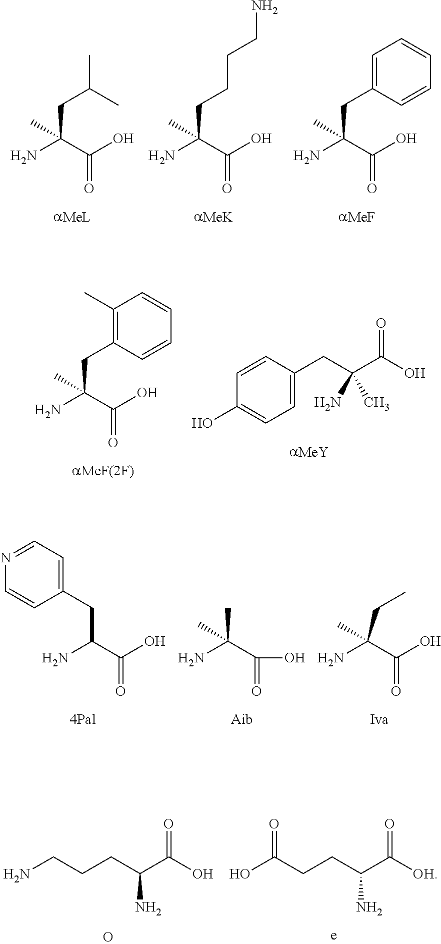

As noted above, the incretin analogs herein have structural similarities to, but many structural differences, from any of the native human peptides. For example, when compared to native human GIP (SEQ ID NO:1), the incretin analogs include modifications at one or more of positions 2-3, 6-7, 13-14, 16-18, 20-21, 23-25 and 28-42. In some instances, the incretin analogs include modifications to the amino acids of native human GIP (SEQ ID NO:1) at each of positions 2-3, 6-7, 13-14, 16-18, 20-21, 23-25 and 28-42. In certain instances, the incretin analogs include the following amino acid modifications: Aib at position 2; Q at position 3; αMeF(2F) at position 6; T at position 7; Y or 4Pal at position 10; L or αMeL at position 13; L at position 14; a modified K residue at position 17, which is modified through conjugation to the epsilon-amino group of the K-side chain with a C₁₆ to C₂₂ fatty acid, optionally through the use of a linker; A at position 18; 4Pal, Iva or αMeL at position 20; A or Aib at position 21; I at position 23; E or e at position 24; Y or αMeY at position 25; E at position 28; G at position 29; and replacement of the amino acids at positions 30-42 with the following amino acid sequence: GPSSGAPPPE (SEQ ID NO:12) or GPSSGAPPPS (SEQ ID NO:13) (and truncated analogs of either of these tails). In yet other instances, the incretin analogs are amidated. In addition to the modifications described herein, the incretin analogs may include one or more additional amino acid modifications such as an addition, deletion, insertion and/or substitution, but remain capable of binding to and activating each of the GIP, GLP-1 and GCG receptors.

As noted above, the incretin analogs herein include a fatty acid moiety conjugated, for example, by way of a linker to a standard or non-standard amino acid with a functional group available for conjugation. Such a conjugation is sometimes referred to as acylation.

In certain instances, the amino acid with a functional group available for conjugation can be C, D, E, K or Q. In particular instances, the amino acid with a functional group available for conjugation is K, where the conjugation is to the epsilon-amino group of the K side-chain.

The acylation of the incretin analogs herein is at position 17 in SEQ ID NO:5, which is determined to be an optimal location for including this structure. The fatty acid moiety, and in certain instances the linker, acts as an albumin binder and provides a potential to generate long-acting compounds.

The incretin analogs herein utilize a C₁₆-C₂₂ fatty acid moiety chemically conjugated to the functional group of an amino acid either by a direct bond or by a linker.

The length and composition of the fatty acid moiety impacts the half-life of the incretin analogs, the potency of the incretin analogs in in vivo animal models, and the solubility and stability of the incretin analogs. Conjugation to a C₁₆-C₂₂ saturated fatty monoacid or diacid results in incretin analogs that exhibit desirable half-life, desirable potency in in vivo animal models, and desirable solubility and stability characteristics.

Examples of saturated C₁₆-C₂₂ fatty acids for use herein include, but are not limited to, palmitic acid (hexadecanoic acid) (C₁₆ monoacid), hexadecanedioic acid (C₁₆ diacid), margaric acid (heptadecanoic acid (C₁₇ monoacid), heptadecanedioic acid (C₁₇ diacid), stearic acid (Cis monoacid), octadecanedioic acid (Cis diacid), nonadecylic acid (nonadecanoic acid (C₁₉ monoacid), nonadecanedioic acid (C₁₉ diacid), arachadic acid (eicosanoic acid (C₂₀ monoacid), eicosanedioic acid (C₂₀ diacid), heneicosylic acid (heneicosanoic acid (C₂₁ monoacid), heneicosanedioic acid (C₂₁ diacid), behenic acid (docosanoic acid (C₂₂ monoacid), docosanedioic acid (C₂₂ diacid), including branched and substituted derivatives thereof.

In certain instances, the C₁₆-C₂₂ fatty acid can be a saturated C₂₀ monoacid, a saturated C₂₀ diacid, and branched and substituted derivatives thereof. In more particular instances, the C₁₆-C₂₂ fatty acid can be eicosanedioic acid.

In some instances, the linker can have from one to four amino acids, an amino polyethylene glycol carboxylate or mixtures thereof. In certain instances, the amino polyethylene glycol carboxylate has the following structure:

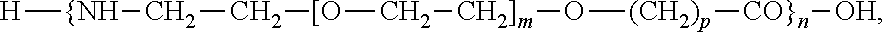

where m is any integer from 1 to 12, n is any integer from 1 to 12, and p is 1 or 2.

In certain instances, the linker can have one or more AEEA moieties, optionally in combination with one to four amino acids.

In instances in which the linker includes at least one amino acid, the amino acid can be one to four E or γE amino acid residues. In some instances, the linker can include one or two E or γE amino acid residues, including the D-forms thereof. For example, the linker can include either one or two γE amino acid residues. Alternatively, the linker can include one to four amino acid residues (such as, for example, E or γE amino acids) used in combination with up to thirty-six AEEA moieties. Specifically, the linker can be combinations of one to four E or γE amino acids and one to four AEEA moieties. In other instances, the linker can be combinations of one or two γE amino acids and one or two AEEA moieties.

In a specific instance, the incretin analogs herein have a linker-fatty acid moiety having the following structure:

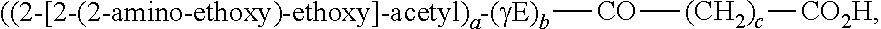

where a is 0, 1 or 2, b is 1 or 2, and c is 16 or 18. In a particular instance, a is 1, bis and c is 20, having the following structure:

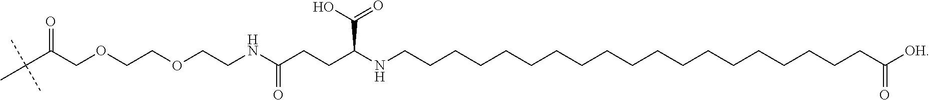

As shown in the chemical structures of Examples 1-3 below, the linker-fatty acid moiety described above can be linked to the epsilon (ε)-amino group of the K side-chain.

The affinity of the incretin analogs herein for each of the GIP, GLP-1 and GCG receptors may be measured using techniques known in the art for measuring receptor binding levels, including, for example, those described in the examples below, and is commonly expressed as an inhibitory constant (Ki) value. The activity of the incretin analogs at each of the receptors also may be measured using techniques known in the art, including, for example, the in vitro activity assays described below, and is commonly expressed as an effective concentration 50 (EC₅₀) value, which is the concentration of compound causing half-maximal simulation in a dose response curve.

The incretin analogs herein can be formulated as pharmaceutical compositions, which can be administered by parenteral routes (e.g., subcutaneous, intravenous, intraperitoneal, intramuscular or transdermal). Such pharmaceutical compositions and techniques for preparing the same are well known in the art. See, e.g., Remington: The Science and Practice of Pharmacy (Troy, Ed., 21^{st} Edition, Lippincott, Williams & Wilkins, 2006). In particular instances, the incretin analogs are administered subcutaneous.

The incretin analogs herein may react with any of several inorganic and organic acids/bases to form pharmaceutically acceptable acid/base addition salts. Pharmaceutically acceptable salts and common techniques for preparing them are well known in the art (see, e.g., Stahl, et al. Handbook of Pharmaceutical Salts: Properties, Selection and Use, 2^{nd} Revised Edition (Wiley-VCH, 2011)). Pharmaceutically acceptable salts for use herein include, but are not limited to, sodium, trifluoroacetate, hydrochloride, and acetate salts.

The disclosure also provides and therefore encompasses novel intermediates and methods of synthesizing the incretin analogs herein, or a pharmaceutically acceptable salt thereof. The intermediates and incretin analogs herein can be prepared by a variety of techniques known in the art. For example, a method using chemical synthesis is illustrated in the Examples below. The specific synthetic steps for each of the routes described may be combined in different ways to prepare incretin analogs herein. The reagents and starting materials are readily available to one of skill in the art.

Certain incretin analogs herein are generally effective over a wide dosage range. For example, dosages for once-weekly administration may fall within a range of about 0.01 to about 30 mg/individual/week, within a range of about 0.1 to about 10 mg/individual/week, or even within a range of about 0.1 to about 3 mg/individual/week. Thus, the incretin analogs herein may be dosed daily, thrice-weekly, twice-weekly or once-weekly, especially once-weekly administration.

The incretin analogs herein may be used for treating a variety of conditions, disorders, diseases or symptoms. In particular, methods are provided for treating T2DM in an individual, where such methods include at least a step of administering to an individual in need of such treatment an effective amount of an incretin analog herein, or a pharmaceutically acceptable salt thereof.

Additionally, methods are provided for treating dyslipidemia in an individual, where such methods include at least a step of administering to an individual in need of such treatment an effective amount of an incretin analog herein, or a pharmaceutically acceptable salt thereof.

Additionally, methods are provided for treating metabolic syndrome in an individual, where such methods include at least a step of administering to an individual in need of such treatment an effective amount of an incretin analog herein, or a pharmaceutically acceptable salt thereof.

Additionally, methods are provided for treating NAFLD in an individual, where such methods include at least a step of administering to a subject in need of such treatment an effective amount of an incretin analog herein, or a pharmaceutically acceptable salt thereof.

Additionally, methods are provided for treating NASH in an individual, where such methods include at least a step of administering to an individual in need of such treatment an effective amount of an incretin analog herein, or a pharmaceutically acceptable salt thereof.

Additionally, methods are provided for treating obesity in an individual, where such methods include at least a step of administering to an individual in need of such treatment an effective amount of an incretin analog herein, or a pharmaceutically acceptable salt thereof.

Additionally, methods are provided for inducing non-therapeutic weight loss in an individual, where such methods include at least a step of administering to an individual in need of such treatment an effective amount of an incretin analog herein, or a pharmaceutically acceptable salt thereof.

Additionally, methods are provided for treating a cardiovascular disease that is not considered a metabolic disease, where such methods include at least a step of administering to an individual in need of such treatment an effective amount of an incretin analog herein, or a pharmaceutically acceptable salt thereof.

Additionally, methods are provided for treating a neurodegenerative disease, where such methods include at least a step of administering to an individual in need of such treatment an effective amount of an incretin analog herein, or a pharmaceutically acceptable salt thereof.

In these methods, effectiveness of the incretin analogs herein can be assessed by, for example, observing a significant reduction in blood glucose, observing a significant increase in insulin, observing a significant reduction in HbA1c, observing a significant decrease in a blood lipid, observing a significant reduction in weigh or body fat and/or observing a change in body composition.

Alternatively, the incretin analogs herein or pharmaceutically acceptable salts thereof may be used for improving bone strength in an individual in need thereof. In some instances, the individual in need thereof has hypoostosis or hypoosteoidosis, or is healing from bone fracture, orthotic procedure, prosthetics implant, dental implant and/or spinal fusion. The incretin analogs also may be used for treating other disorders such as Alzheimer's disease or Parkinson's disease.

### EXAMPLES

The following non-limiting examples are offered for purposes of illustration, not limitation.

### Polypeptide Synthesis

### Example 1: Synthesis of Incretin Analog 1

Example 1 is a compound represented by the following description:

(SEQ ID NO: 9)
YAibQGT-αMeF(2F)-TSDYSILLDOK(2-[2-(2-amino-
ethoxy)-ethoxy]-acetyl-γE-CO—(CH₂)₁₈—CO₂H)AQ-
4Pal-AFIEYLLEGGPSSGEPPPE-NH₂.

Below is a depiction of the structure of Example 1 using the standard single letter amino acid codes except for residues Aib2, αMeF(2F)6, O16, K17, 4Pal20 and E39 where the structures of these amino acid residues have been expanded:

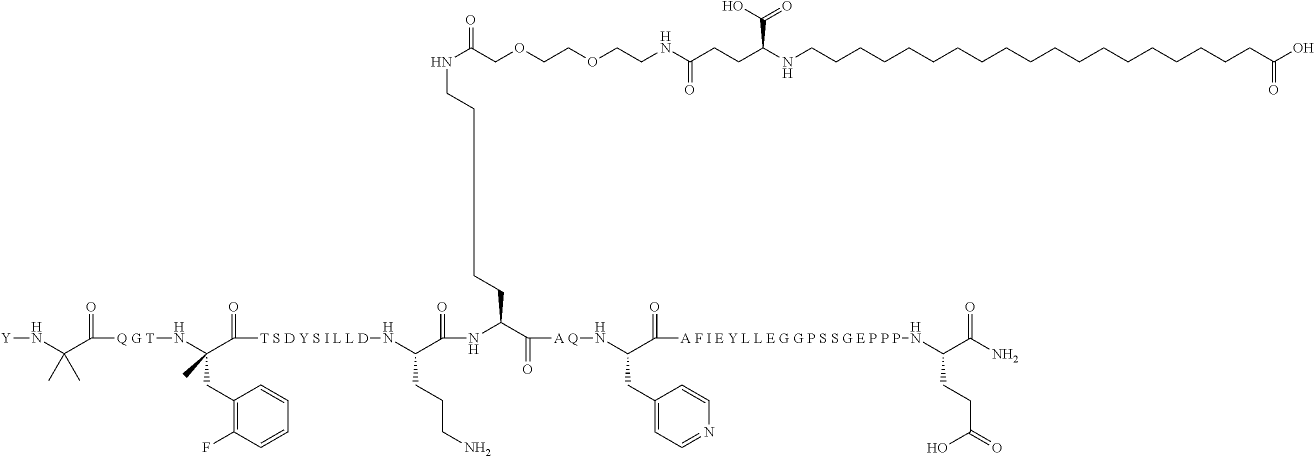

The peptide backbone of Example 1 is synthesized using Fmoc/t-Bu chemistry on a Symphony X peptide synthesizer (Gyros Protein Technologies; Tucson, AZ).

The resin is 1% DVB cross-linked polystyrene (Fmoc-Rink-MBHA low-loading resin, 100-200 mesh; EMD Millipore) at a substitution of 0.3-0.4 meq/g. Standard side-chain protecting groups are used with the following exceptions. Fmoc-Lys(Mtt)-OH is used for the K at position 17, and Boc-Tyr(tBu)-OH) is used for the Y at position 1. Fmoc groups are removed prior to each coupling step (2×7 min) using 20% piperidine in DMF. All standard amino acid couplings are performed for 1 hr to a primary amine and for 3 hr to a secondary amine using an equal molar ratio of Fmoc amino acid (0.3 mM), diisopropylcarbodiimide (0.9 mM) and Oxyma (0.9 mM), at a nine-fold molar excess over the theoretical peptide loading. Exceptions are couplings to Ca-methylated amino acids, which are coupled for 3 hr. After completing synthesis of the peptide backbone, the resin is thoroughly washed with DCM for six times to remove residual DMF. The Mtt protecting group on the K at position 17 is selectively removed from the peptide resin using two treatments of 30% hexafluoroisopropanol (Oakwood Chemicals) in DCM (2×40-minute treatment).

Subsequent attachment of the fatty acid-linker moiety is accomplished by coupling 2-[2-(2-Fmoc-amino-ethoxy)-ethoxy]-acetic acid (Fmoc-AEEA-OH, ChemPep, Inc.), Fmoc-glutamic acid α-t-butyl ester (Fmoc-E-OtBu, Ark Pharm, Inc.) and mono-OtBu-eicosanoic acid (WuXi AppTec, Shanghai, China). A three-fold excess of reagents (AA:PyAOP:DIPEA=1:1:1 mol/mol) is used for each coupling for 1 hr.

After synthesis is complete, the peptide resin is washed with DCM and then thoroughly air-dried. The dry resin is treated with 10 mL of a cleavage cocktail (TFA:triisopropylsilane:water, 92.5:2.5:5 by volume) for 30 min at 40° C. The resin is filtered off, washed twice each with 2 mL of neat TFA, and the combined filtrates are treated with five-fold excess volume of cold diethyl ether (−20° C.) to precipitate the crude peptide. The peptide/ether suspension is then centrifuged at 3500 rpm for 2 min to form a solid pellet, the supernatant is decanted, and the solid pellet is triturated with cold ether two additional times and dried in vacuo. The crude peptide is solubilized in 20% acetonitrile/20% acetic acid/60% water and purified by RP-HPLC on a Luna 5 μm Phenyl-Hexyl preparative column (21×250 mm; Phenomenex) with linear gradients of 100% acetonitrile and 0.1% TFA/water buffer system(30-50% acetonitrile in 60 min). The purity of peptide is assessed using analytical RP-HPLC and pooling criteria is >95%. The main pool purity of Example 1 is found to be >98.0%. Subsequent lyophilization of the final main product pool yields the lyophilized peptide TFA salt. The molecular weight is determined by LC-MS (obsd: M+3=1633.7; Calc M+3=1633.8).

### Example 2: Synthesis of Incretin Analog 2

Example 2 is a compound represented by the following description:

(SEQ ID NO: 10)
YAibQGT-αMeF(2F)-TSDYSI-αMeL-LDOK(2-[2-(2-amino-
ethoxy)-ethoxy]-acetyl-γE-CO—(CH₂)₁₈—CO₂H)AQ-
Iva-AFIE-αMeY-LLEGGPSSGEPPPS-NH₂.

Below is a depiction of the structure of Example 2 using the standard single letter amino acid codes except for residues Aib2, αMeF(2F)6, cMeL13, O16, K17, Iva20, αMeY25 and S39 where the structures of these amino acid residues have been expanded:

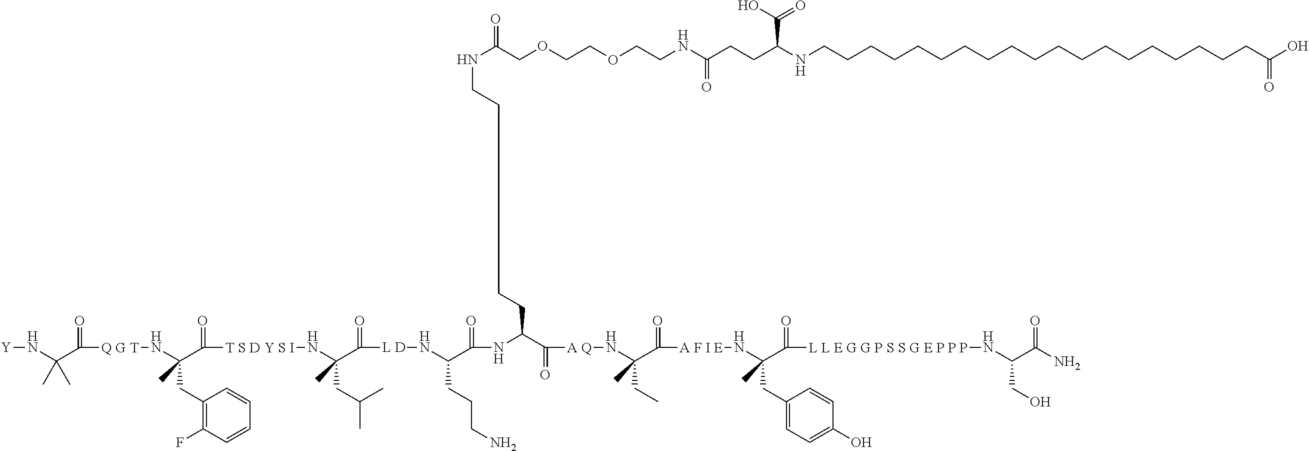

Similar processes to those described above for Example 1 are used to synthesize the peptide backbone, to conjugate the fatty acid-linker moiety, to examine the purity, and to confirm the molecular weight of Example 2.

### Example 3: Synthesis of Incretin Analog 3

Example 3 is a compound represented by the following description:

(SEQ ID NO: 11)
YAibQGT-αMeF(2F)-TSD-4Pal-SI-αMeL-LDOK(2-[2-(2-
amino-ethoxy)-ethoxy]-acetyl-γE-CO—(CH₂)₁₈—
CO₂H)AQ-αMeL-Aib-FIe-αMeY-LLEGGPSSGEPPPS-NH₂.

Below is a depiction of the structure of Example 3 using the standard single letter amino acid codes except for residues Aib2, αMeF(2F)6, 4Pal10, αMeL13, O16, K17, αMeL20, Aib21, e24, αMeY25 and S39 where the structures of these amino acid residues have been expanded:

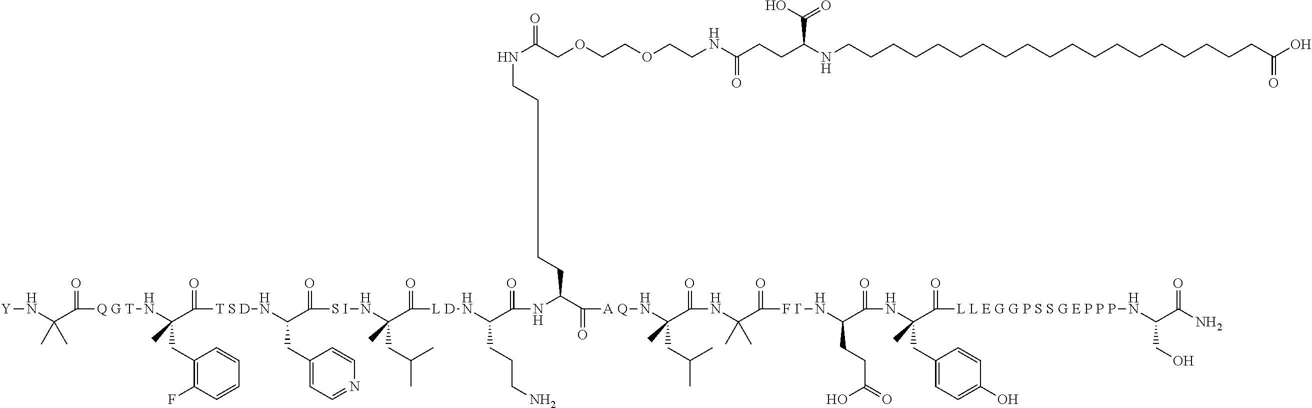

Similar processes to those described above for Example 1 are used to synthesize the peptide backbone, to conjugate the fatty acid-linker moiety, to examine the purity, and to confirm the molecular weight of Example 3.

### In Vitro Function

### Example 4: Binding Affinity

Radioligand competition binding assays are used to determine the equilibrium dissociation constant for the Example compounds and comparators. Such assays use SPA methods and membranes prepared from transfected HEK293 cells overexpressing human GIP receptor (hGIPR; SEQ ID NO:14), human GLP-1 receptor (hGLP-1R; SEQ ID NO:15) or human GCG receptor (hGCGR; SEQ ID NO:16).

The assays are performed in the presence of bacitracin as anon-specific blocking agent to prevent acylated moieties of test analogs from binding to protein components used in standard assay buffers (e.g., albumin).

Competition curves are plotted as the percent specific inhibition (y-axis) versus log concentration of compound (x-axis) and analyzed using a four-parameter nonlinear regression fit with variable slope (ABase or Genedata). Kᵢ values are calculated according to the equation Kᵢ=IC₅₀/(1+(D/K_{d})), where IC₅₀ is the concentration of compound resulting in 50% inhibition of binding, D is the concentration of radioligand used in the assay, and K_{d} is the equilibrium dissociation constant for the receptor and the radioligand, determined from saturation binding analysis (shown in Table 1 below).

TABLE 1
K_{d} Determined from Saturation Binding Analysis.
K_{d}, nM

| hGIPR | hGLP-1R | hGCGR |
|---|---|---|
| 0.14 | 1.2 | 3.9 |

Kᵢ values of Example compounds and comparators are shown in Table 2.

TABLE 2
In Vitro Kᵢ of Example Compounds and Comparators
for hGIPR, hGLP-1R and hGCGR.

| | Kᵢ, nM (SEM, n) | | | | | |
|---|---|---|---|---|---|---|
| Compound | hGIPR | | hGLP-1R | | hGCGR | |
| hGIP | 0.186 | (0.001,101) | — | | — | |
| hGLP-1 | — | | 0.844 | (0.023, 93) | — | |
| hGCG | — | | — | | 3.00 | (0.12, 76) |
| Example 1 | 0.111 | (0.021, 7) | 4.53 | (0.855, 7) | 0.629 | (0.0415, 7) |
| Example 2 | 0.158 | (0.029, 7) | 2.54 | (0.501, 7) | 0.950 | (0.350, 7) |
| Example 3 | 0.114 | (0.007, 5) | 3.63 | (0.386, 5) | 1.73 | (0.296, 5) |

NOTE:
A qualifier (>) indicates the data did not reach 50% inhibition relative to maximum binding, whereby the Kᵢ was calculated using the highest concentration tested in the assay. n = l/x means that only one value out of the total number of replicates (x) is used to express the mean. SEM is only calculated when n = 2 or greater non-qualified results exist.

As seen in Table 2, the Example compounds have binding affinity at each of the hGIPR, hGLP-1R and hGCGR.

### Example 5: Functional hGIPR, hGLP-1R and hGCGR Assays

Methods: Functional activity is determined using cAMP formation in HEK-293 clonal cell lines expressing hGIPR, hGLP-1R or hGCGR hGIPR, hGLP-1R or hGCGR receptor-expressing cells are treated with a control polypeptide or one of Examples 1 to 3 (20 point concentration-response curve in DMSO, 2.75-fold Labcyte Echo direct dilution, 384 well plate Corning Cat #3570) in DMEM (Gibco Cat #31053) supplemented with 1× GlutaMAX™ (Gibco Cat #35050), 0.1% bovine casein (Sigma C4765-10ML), 250 μM IBMX (3-Isobutyl-1-methylxanthine, Acros Cat #228420010) and 20 mM HEPES (Gibco Cat #15630) in a 20 μL assay volume (final DMSO concentration was 0.5%). Experiments also are performed under identical assay conditions with the addition of 1.0% fatty acid free, globulin free human serum albumin (Sigma Cat #A3782).

After a 30-min incubation at 37° C., the resulting increase in intracellular cAMP is quantitatively determined using a CisBio cAMP Dynamic 2 HTRF Assay Kit (62AM4PEJ). Briefly, cAMP levels within the cell are detected by adding the cAMP-d2 conjugate in cell lysis buffer (10 μL) followed by the antibody anti-cAMP-Eu³⁺-Cryptate, also in cell lysis buffer (10 μL). The resulting competitive assay is incubated for at least 60 min at room temperature, and then is detected using a PerkinElmer Envision® instrument with excitation at 320 nm and emission at 665 nm and 620 nm. Envision units (emission at 665 nm/620 nm*10,000) are inversely proportional to the amount of cAMP present and are converted to nM cAMP per well using a cAMP standard curve. The amount of cAMP generated (nM) in each well is converted to a percent of the maximal response observed with human GIP(1-42)NH₂, hGLP-1(7-36)NH₂ or hGCG. A relative EC₅₀ value and percent top (Eₘₐₓ) are derived by non-linear regression analysis using the percent maximal response vs. the concentration of peptide added, fitted to a four-parameter logistic equation.

Results: Functional data for hGIP(1-42)NH₂, hGLP-1(7-36)NH₂, hGCG and the Example compounds are provided below in Table 3 (0.1% bovine casein) and Table 4 (0.1% bovine casein, 1.0% human serum albumin).

TABLE 3
Functional cAMP Potency (EC₅₀) and Efficacy (Eₘₐₓ) for Peptides
Incubated at 37° C. (in the presence of 0.1% bovine casein).

| | hGLP-1R ^{a} | | hGCGR ^{a} | | hGIPR ^{a} | |
|---|---|---|---|---|---|---|
| | EC₅₀, nM, | Eₘₐₓ, | EC₅₀, nM, | Eₘₐₓ, | EC₅₀, nM, | Eₘₐₓ, |
| Compound | SEM (n) ^{b} | % ± SEM ^{c} | SEM (n) ^{b} | % ± SEM ^{c} | SEM (n) ^{b} | % ± SEM ^{c} |
| hGLP-1 | 0.395 | 103 ± 2 | — | — | — | — |
| (7-36)NH₂ | 0.032 (62) | | | | | |
| hGCG | — | — | 2.17 | 103 ± 2 | — | — |
| | | | 0.17 (63) | | | |
| hGIP | — | — | — | — | 1.08 | 98 ± 2 |
| (1-42)NH₂ | | | | | 0.14 (60) | |
| Example 1 | 0.345 | 106 ± 4 | 1.20 | 101 ± 3 | 0.0513 | 102 ± 2 |
| | 0.033 (10) | | 0.12 (10) | | 0.0099 (10) | |
| Example 2 | 0.383 | 103 ± 3 | 1.02 | 103 ± 2 | 0.0256 | 101 ± 2 |
| | 0.089 (10) | | 0.19 (10) | | 0.0052 (10) | |
| Example 3 | 0.451 | 107 ± 2 | 2.49 | 99 ± 4 | 0.0306 | 103 ± 2 |
| | 0.079 (11) | | 0.37 (10) | | 0.0061 (10) | |

NOTE:
^{a} Expression density is determined using homologous competition binding of [¹²⁵I]GLP-1(7-36)NH₂ at hGLP-1R (112 fmol/mg protein), [¹²⁵I]GCG at hGCGR (98 fmol/mg protein) and [¹²⁵I]GIP(1-42) at hGIPR (124 fmol/mg protein),
^{b} EC₅₀, nM = the Geometric Mean with the Standard Error of the Mean followed by the number of observations in parenthesis.
^{c} Eₘₐₓ, % = the Arithmetic Mean ± the Standard Error of the Mean for the percent of maximal response to GLP-1(7-36)NH₂ at hGLP-1R, GCG at hGCGR or GIP(1-42)NH₂ at hGIPR. All values shown are to three (3) significant digits.

TABLE 4
Functional cAMP Potency (EC₅₀) and Efficacy (Eₘₐₓ) for Peptides Incubated at
37° C. (in the presence of 0.1% bovine casein and 1.0% human serum albumin).

| | hGLP-1R ^{a} | | hGCGR ^{a} | | hGIPR ^{a} | |
|---|---|---|---|---|---|---|
| | EC₅₀, nM, | Eₘₐₓ, | EC₅₀, nM, | Eₘₐₓ, | EC₅₀, nM, | Eₘₐₓ, |
| Compound | SEM (n) ^{b} | % ± SEM ^{c} | SEM (n) ^{b} | % ± SEM ^{c} | SEM (n) ^{b} | % ± SEM ^{c} |
| hGLP-1 | 0.368 | 104 ± 3 | — | — | — | — |
| (7-36)NH₂ | 0.030 (45) | | | | | |
| hGCG | — | — | 2.54 | 102 ± 3 | — | — |
| | | | 0.30 (45) | | | |
| hGIP | — | — | — | — | 0.756 | 96 ± 2 |
| (1-42)NH₂ | | | | | 0.090 (45) | |
| Example 1 | 180 | 102 ± 4 | 899 | 103 ± 8 | 22.3 | 100 ± 3 |
| | 17 (7) | | 118 (6) | | 5.4 (7) | |
| Example 2 | 119 | 116 ± 4 | 537 | 112 ± 10 | 2.91 | 99 ± 2 |
| | 15 (7) | | 99 (8) | | 0.46 (7) | |
| Example 3 | 114 | 111 ± 5 | 480 | 97 ± 9 | 3.72 | 105 ± 5 |
| | 12 (6) | | 178 (6) | | 0.83 (7) | |

NOTE:
^{a} Expression density is determined using homologous competition binding of [¹²⁵I]GLP-1(7-36)NH₂ at hGLP-1R (112 fmol/mg protein), [¹²⁵I]GCG at hGCGR (98 fmol/mg protein) and [¹²⁵I]GIP(1-42) at hGIPR (124 fmol/mg protein).
^{b} EC₅₀, nM = the Geometric Mean with the Standard Error of the Mean followed by the number of observations in parenthesis.
^{c} Eₘₐₓ, % = the Arithmetic Mean ± the Standard Error of the Mean for the percent of maximal response to GLP-1(7-36)NH₂ at hGLP-1R, GCG at hGCGR or GIP(1-42)NH₂ at hGIPR. All values shown are to three (3) significant digits.

As seen in Table 3, Example compounds stimulate cAMP from hGLP-1R, hGCGR and hGIPR in the presence of 0.1% casein.

As seen in Table 4, Example compounds stimulate cAMP from hGLP-1R, hGCGR and hGIPR in the presence of 0.1% bovine casein and 1% human serum albumin.

### In Vivo Function

### Example 6: Pharmacokinetics in Male Diet-Induced Obese (DIO) Mice

Methods: Male DIO mice are administered a single SQ dose of 200 nmol/kg (0.98 mg/kg) of Example compounds in 40 mM Tris-HCL pH8 buffer with 0.02% PS80 at a volume of 10 mL/kg. Blood is collected at 1, 3, 6, 12, 24, 48 and 72 hr post-dose for pharmacokinetic characterization. Plasma concentration of Example compounds are determined by a qualified Liquid Chromatography Mass Spectrometry (LC/MS) method at Q Squared Solutions BioSciences LLC (Ithaca, NY). The Example compounds and an internal standard are extracted from 100% mouse plasma using protein precipitation followed by solid phase extraction. The intact mass of the Example compounds, which includes peptide plus acyl chain, are detected by a Q-Exactive™ Orbitrap® mass spectrometer.

Results: Data for the Example compounds are provided below in Table 5.

TABLE 5
Mean Plasma Pharmacokinetic Parameters Following
a Single 200 nmol/kg SQ Dose to Male DIO Mice.

| | t½ | Tₘₐₓ | Cₘₐₓ | AUC_{0-inf} | CL/F |
|---|---|---|---|---|---|
| Compound | (hr) | (hr) | (nmol/L) | (hr*nmol/L) | (mL/hr/kg) |
| Example 1 | 38 | 12 | 817 | 55550 | 3.6 |
| Example 2 | 18 | 12 | 1239 | 42479 | 4.7 |
| Example 3 | 13 | 12 | 1072 | 32800 | 6.1 |

NOTE:
Abbreviations:
t½ = half-life,
Tₘₐₓ = time to maximum concentration,
Cₘₐₓ = maximum observed plasma concentration,
AUC_{0-inf} = area under the curve from time 0 hours to infinity,
CL/F = clearance/bioavailability.
N = 3 animals/group/time point.

As seen in Table 5, the Example compounds demonstrate an extended pharmacokinetic profile in DIO mice.

### Example 7: In Vivo Effect on Insulin Secretion in Male Wistar Rats

Methods: An intravenous glucose tolerance test (ivGTT) in Male Wistar rats is used to estimate insulinotropic potency of the Example compounds. A GLP-1R agonist, semaglutide, is used as a positive control. Rats with surgically implanted cannulas in the jugular vein and carotid artery (Envigo; Indianapolis, IN) 280-320 g are housed one per cage in polycarbonate cages with filter tops. Rats are maintained on a 12-hr light-dark cycle at 21° C. and receive 2014 Teklad Global diet (Envigo) and deionized water ad libitum. Rats are randomized by body weight and dosed 1.5 mL/kg SQ with Example compounds 16 hr prior to glucose administration and then fasted. Stock concentrations of 211 nM/mL of Example compounds are diluted in 40 mM Tris-HCl buffer pH 8.0 with 0.02% PS80 to desired dosing concentrations; doses tested are vehicle, 0.1, 0.3, 1, 3, 10 and 30 nM/kg. Semaglutide is used as positive control in connection with each run of Example compounds (10 nM/kg dose).

A time 0 blood sample is collected into EDTA tubes after which glucose is administered (0.5 mg/kg, 5 mL/kg). Blood samples are collected for glucose and insulin levels at time 2, 4, 6, 10, 20 and 30 min post intravenous administration of glucose. Plasma insulin is determined using an electrochemiluminescence assay (Meso Scale; Rockville, MD). Insulin AUC is examined compared to the vehicle control with n=6 animals per group.

Statistical analysis is performed using JMP with a one-way ANOVA followed by Dunnett's comparison to the vehicle control.

Results: Data for the Example compounds are provided below in Table 6.

TABLE 6
Effect of Vehicle, Semaglutide (Sema at 10 nmol/kg) and Example
Compounds on Insulin Secretion During ivGTT in Wistar Rats.

| | AUC_{30 min} of Insulin After Bolus IV Glucose | | | | | | | |
|---|---|---|---|---|---|---|---|---|
| Com- | Dose (nmol/kg) | | | | | | | |
| pound | Vehicle | 0.1 | 0.3 | 1 | 3 | 10 | 30 | Sema |
| Ex- | 27.8 ± | 36.1 ± | 45.5 ± | 70.4 ± | 115.7 ± | 145.9 ± | 134.5 ± | 80.9 ± |
| ample | 4.8 | 5.0 | 8.4 | 8.4 | 6.0 | 14.9 | 16.6 | 8.2 |
| 1 | | | | | | | | |
| Ex- | 28.9 ± | 69.0 ± | 62.6 ± | 72.6 ± | 101.5 ± | 128.8 ± | 159.0 ± | 76.4 ± |
| ample | 7.2 | 7.4 | 5.1 | 4.7 | 18.2 | 11.7 | 14.9 | 5.6 |
| 2 | | | | | | | | |
| Ex | 37.3 ± | 67.0 ± | 77.6 ± | 86.9 ± | 97.2 ± | 119.8 ± | 174.8 ± | 76.6 ± |
| ample | 4.1 | 10.2 | 6.7 | 9.1 | 7.5 | 10.2 | 22.1 | 7.1 |
| 3 | | | | | | | | |

NOTE:
Results are expressed as Mean ± Standard Error of Means (SEM) of 6 rats per group. The statistical test is one-way ANOVA followed by Dunnett's
*p < 0.05 compared to vehicle;
+p < 0.05 compared to semaglutide.

As seen in Table 6, the Example compounds dose-dependently increase insulin secretion.

### Example 8: In Vivo Studies on Weight Loss, Metabolism and Body Composition in DIO C57/B16 Mice

Methods: To investigate the effects of the Example compounds on parameters such as weight loss, metabolism and body composition, the Example compounds are dosed to C57B16 DIO mice. These animals, although not diabetic, display insulin resistance and dyslipidemia, all characteristics of metabolic syndrome, after being placed on a high fat diet for 20 weeks.

Here, DIO male C57/B16 mice at 20 weeks old are maintained on a calorie-rich diet are used in the following studies. Animals are individually housed in a temperature-controlled (23° C. to 26° C.) facility with 12-hr light/dark cycle (lights on 2100) and free access to food (TD95217) and water. After a 3-week acclimation to the facility, which includes 1 week of vehicle acclimation dosing, the mice are randomized according to their body weight, so each experimental group of animals would have similar body weight. The body weights range from 40 g to 51 g.

All groups contain 6 mice. Example compounds and semaglutide are dissolved in vehicle (40 mM Tris-HCl at pH 8.0 with 0.02% PS-80) and are administered by SQ injection (10 mL/kg) to ad libitum-fed DIO mice 30 min to 120 min prior to the onset of the dark cycle every day for 14 days (Days 1 to 14). Body weight and food intake are measured daily throughout the study, including 1 day after last dose (Day 15).

Absolute changes in body weight are calculated by subtracting the body weight of the same animal prior to the first injection of molecule. On Days 0 and 15, total fat mass is measured by nuclear magnetic resonance (NMR) using an Echo Medical System (Houston, TX) instrument. On Day 15, metabolic parameters are measured using Accu-Chek® Aviva® Glucometers (Roche; Indianapolis, IN) or a Hitachi clinical blood analyzer (Roche, Indianapolis, IN). Plasma insulin is determined using an electrochemiluminescence assay (Meso Scale Discovery, Rockville, MD).

Data are presented as mean±t SEM of 6animals per group in Tables 7 and 8 below. Statistical analysis is performed using repeated measures ANOVA, followed by Dunneti's method for multiple comparisons. Significant differences are identified as * with p<0.05.

Results: Data for the Example compounds are provided below in Tables 7 and 8.

TABLE 7
Body Weight Change After Treatment with Semaglutide
and Example Compounds After 15 Days.

| | Dose | Δ Body | Δ Body | Fat Mass |
|---|---|---|---|---|
| Compound | (nmol/kg) | Weight (g) | Weight (%) | (%) |
| Sema | 10 | −9.23 ± 0.58* | −21.98 ± 2.58* | −15.14 ± 1.93* |
| Example 1 | 0.15625 | −1.20 ± 0.15 | −4.31 ± 0.75 | −2.44 ± 0.63 |
| | 0.625 | −4.37 ± 0.08* | −11.40 ± 0.92* | −7.38 ± 1.05* |
| | 2.5 | −10.42 ± 1.40* | −24.43 ± 3.84* | −17.05 ± 2.47* |
| | 10 | −18.97 ± 0.81* | −43.71 ± 2.32* | −30.07 ± 1.69* |
| | 20 | −21.30 ± 0.94* | −48.88 ± 2.00* | −32.15 ± 1.93* |
| Example 2 | 0.15625 | −1.72 ± 0.04 | −2.65 ± 0.55 | −1.91 ± 0.84 |
| | 0.625 | −4.48 ± 0.26* | −8.90 ± 1.38* | −5.87 ± 0.94* |
| | 2.5 | −8.62 ± 0.21* | −17.90 ± 1.01* | −11.96 ± 0.88* |
| | 10 | −20.10 ± 0.54* | −43.44 ± 1.30* | −28.53 ± 1.24* |
| | 20 | −21.77 ± 0.50* | −46.79 ± 1.40* | −28.80 ± 0.81* |
| Example 3 | 0.15625 | −1.95 ± 0.13 | −4.60 ± 0.71 | −3.34 ± 0.60 |
| | 0.625 | −5.18 ± 0.25* | −11.91 ± 0.99* | −8.03 ± 0.84* |
| | 2.5 | −11.65 ± 1.68* | −25.98 ± 3.85* | −18.55 ± 2.41* |
| | 10 | −18.43 ± 0.68* | −41.10 ± 1.51* | −28.50 ± 1.55* |
| | 20 | −19.92 ± 0.34* | −44.22 ± 1.01* | −29.23 ± 1.29* |

NOTE:
“Δ Body weight (g)” refers to difference between body weight at day 15 between test and vehicle groups. “Δ Body weight (%)” refers to percent decrease in body weight between days 1 and 15 in test groups. “Fat mass (%)” refers to difference between fat mass between days 0 and 15 in test groups. “Sema” means semaglutide. All data is from single representative study. Percent change in body weight and fat mass for animals receiving vehicle is recorded and is less than 2% in each study. The “Δ Body weight (g)”, “Δ Body weight (%)”, and “Δ Fat mass (%)” and are statistically significantly different (*, p < 0.05) than control for semaglutide and all Examples at all doses tested, except for Example peptides at the low dose, 0.15625 nmol/kg.

As seen in Table 7, the Example compounds dose-dependently reduce body

TABLE 8
Effect of Treatment with Example Compounds on Blood Glucose,
Insulin, Total Cholesterol and Triglycerides After 15 Days.

| | Dose | Δ Glucose | Δ Insulin | Δ Cholesterol | Δ Trigs |
|---|---|---|---|---|---|
| Compound | (nmol/kg) | (mg/dL) | (ng/mL) | (mg/dL) | (mg/dL) |
| Sema | 10 | −18.33 ± 2.81 | −4.15 ± 2.23 | −73.33 ± 4.17* | 16.00 ± 0.38 |
| Example 1 | 0.15625 | −18.58 ± 1.04 | −4.48 ± 1.70 | −14.33 ± 4.37 | −27.83 ± 0.44 |
| | 0.625 | −24.42 ± 4.44* | −6.03 ± 0.94 | −39.17 ± 0.08 | 15.33 ± 2.75 |
| | 2.5 | −14.67 ± 0.77 | −10.13 ± 4.21 | −118.17 ± 1.57* | −2.83 ± 4.13 |
| | 10 | −35.08 ± 1.47* | −11.14 ± 4.41* | −164.00 ± 1.85* | −43.00 ± 0.50 |
| | 20 | −56.00 ± 2.12* | −11.40 ± 4.55* | −179.17 ± 7.73* | −52.50 ± 3.95* |
| Example 2 | 0.15625 | −9.50 ± 5.21 | 14.64 ± 3.28 | −22.00 ± 4.71 | 9.67 ± 7.80 |
| | 0.625 | −6.17 ± 7.12 | −9.35 ± 5.61 | −43.50 ± 3.47* | −3.50 ± 3.73 |
| | 2.5 | −13.50 ± 3.92 | −14.01 ± 7.49 | −150.33 ± 1.70* | −2.33 ± 3.07 |
| | 10 | −41.00 ± 4.18* | −14.52 ± 7.39 | −199.33 ± 7.02* | −9.00 ± 5.09 |
| | 20 | −46.08 ± 4.21* | −14.73 ± 7.52 | −198.00 ± 5.46* | −15.83 ± 6.63 |
| Example 3 | 0.15625 | −26.97 ± 6.52* | 0.48 ± 1.80 | −14.50 ± 5.03 | 40.67 ± 1.75 |
| | 0.625 | −19.47 ± 6.65* | −4.49 ± 3.03 | −37.83 ± 4.80* | 40.33 ± 1.58 |
| | 2.5 | −33.47 ± 1.69* | −7.25 ± 3.60* | −136.50 ± 5.42* | −7.17 ± 6.52 |
| | 10 | −48.72 ± 6.41* | −7.61 ± 3.80* | −162.50 ± 4.09* | 29.00 ± 21.71 |
| | 20 | −55.97 ± 6.74* | −7.69 ± 3.79* | −186.83 ± 7.59* | −16.50 ± 1.60 |

NOTE:
All data refers to difference at day 15 between test and vehicle groups and is from a single representative study. “Sema” means semaglutide. *p < 0.05 compared to Vehicle group; one-way ANOVA, Dunnett's.

As seen in Table 8, the Example compounds also reduce blood glucose, insulin (as a sign of increasing insulin sensitivity), cholesterol and triglycerides.

### SEQUENCE LISTING

The following nucleic and/or amino acid sequences are referred to in the disclosure above and are provided below for reference.

SEQ ID NO: 1-Human GIP
YAEGTFISDYSIAMDKIHQQDFVNWLLAQKGKKNDWKHNITQ
SEQ ID NO: 2-Human GLP-1₇₋₃₆ amide
HAEGTFTSDVSSYLEGQAAKEFIAWLVKGR-NH₂
SEQ ID NO: 3-Human glucagon
HSQGTFTSDYSKYLDSRRAQDFVQWLMNT
SEQ ID NO: 4-Human OXM
HSQGTFTSDYSKYLDSRRAQDFVQWLMNTKRNRNNIA
SEQ ID NO: 5-Incretin analog
YX₂QGTX₆TSDX₁₀SIX₁₃LDX₁₆X₁₇AQX₂₀X₂₁FIX₂₄X₂₅LLEGGPSSGEPPPX₃₉,
where:
X₂ is Aib,
X₆ is αMeF(2F),
X₁₀ can be Y or 4Pal,
X₁₃ can be L or αMeL,
X₁₆ is Orn,
X₁₇ is any amino acid with a functional group available for conjugation,
X₂₀ can be 4Pal, Iva or αMeL,
X₂₁ can be A or Aib,
X₂₄ can be E or e,
X₂₅ can be Y or αMeY, and
X₃₉ can be E or S
SEQ ID NO: 6-Incretin analog
Y-Aib-QGT-αMeF(2F)-TSDYSILLDOKAQ-4Pal-AFIEYLLEGGPSSGEPPPE
SEQ ID NO: 7-Incretin analog
Y-Aib-QGT-αMeF(2F)-TSDYSI-αMeL-LDOKAQ-Iva-AFIE-αMeY-
LLEGGPSSGEPPPS
SEQ ID NO: 8-Incretin analog
Y-Aib-QGT-αMeF(2F)-TSD-4Pal-SI-αMeL-LDOKAQ-αMeL-Aib-FIe-αaMeY-
LLEGGPSSGEPPPS
SEQ ID NO: 9-Incretin analog
Y-Aib-QGT-αMeF(2F)-TSDYSILLDOK(2-[2-(2-amino-ethoxy)-ethoxy]-acetyl-γE-CO-
(CH₂)₁₈-CO₂H)AQ-4Pal-AFIEYLLEGGPSSGEPPPE-NH₂
SEQ ID NO: 10-Incretin analog
Y-Aib-QGT-αMeF(2F)-TSDYSI-αMeL-LDOK(2-[2-(2-amino-ethoxy)-ethoxy]-acetyl-
γE-CO-(CH₂)₁₈-CO₂H)AQ-Iva-AFIE-αMeY-LLEGGPSSGEPPPS-NH₂
SEQ ID NO: 11-Incretin analog
Y-Aib-QGT-αMeF(2F)-TSD-4Pal-SI-αMeL-LDOK(2-[2-(2-amino-ethoxy)-ethoxy]-
acetyl-γE-CO-(CH₂)₁₈-CO₂H)AQ-αMeL-Aib-FIe-αMeY-LLEGGPSSGEPPPS-NH₂
SEQ ID NO: 12-Artificial sequence
GPSSGEPPPE
SEQ ID NO: 13-Artificial sequences
GPSSGEPPPS
SEQ ID NO: 14-human GIP receptor
MTTSPILQLLLRLSLCGLLLQRAETGSKGQTAGELYQRWERYRRECQETLAAAEP
PSGLACNGSFDMYVCWDYAAPNATARASCPWYLPWHHHVAAGFVLRQCGSDG
QWGLWRDHTQCENPEKNEAFLDQRLILERLQVMYTVGYSLSLATLLLALLILSLF
RRLHCTRNYIHINLFTSFMLRAAAILSRDRLLPRPGPYLGDQALALWNQALAACR
TAQIVTQYCVGANYTWLLVEGVYLHSLLVLVGGSEEGHFRYYLLLGWGAPALF
VIPWVIVRYLYENTQCWERNEVKAIWWIIRTPILMTILINFLIFIRILGILLSKLRTRQ
MRCRDYRLRLARSTLTLVPLLGVHEVVFAPVTEEQARGALRFAKLGFEIFLSSFQ
GFLVSVLYCFINKEVQSEIRRGWHHCRLRRSLGEEQRQLPERAFRALPSGSGPGE
VPTSRGLSSGTLPGPGNEASRELESYC
SEQ ID NO: 15-human GLP-1 receptor
MAGAPGPLRLALLLLGMVGRAGPRPQGATVSLWETVQKWREYRRQCQRSLTED
PPPATDLFCNRTFDEYACWPDGEPGSFVNVSCPWYLPWASSVPQGHVYRFCTAE
GLWLQKDNSSLPWRDLSECEESKRGERSSPEEQLLFLYIIYTVGYALSFSALVIAS
AILLGFRHLHCTRNYIHLNLFASFILRALSVFIKDAALKWMYSTAAQQHQWDGLL
SYQDSLSCRLVFLLMQYCVAANYYWLLVEGVYLYTLLAFSVLSEQWIFRLYVSI
GWGVPLLFVVPWGIVKYLYEDEGCWTRNSNMNYWLIIRLPILFAIGVNFLIFVRVI
CIVVSKLKANLMCKTDIKCRLAKSTLTLIPLLGTHEVIFAFVMDEHARGTLRFIKL
FTELSFTSFQGLMVAILYCFVNNEVQLEFRKSWERWRLEHLHIQRDSSMKPLKCP
TSSLSSGATAGSSMYTATCQASCS
SEQ ID NO: 16-human GCG receptor
MPPCQPQRPLLLLLLLLACQPQVPSAQVMDFLFEKWKLYGDQCHHNLSLLPPPTL
VCNRTFDKYSCWPDTPANTTANISCPWYLPWHHKVQHRFVFKRCGPDGQWVRG
PRGQPWRDASQCQMDGEEIEVQKEVAKMYSSFQVMYTVGYSLSLGALLLALAIL
GGLSKLHCTRNAIHANLFASFVLKASSVLVIDGLLRTRYSQKIGDDLSVSTWLSD
GAVAGCRVAAVFMQYGIVANYCWLLVEGLYLHNLLGLATLPERSFFSLYLGIG
WGAPMLFVVPWAVVKCLFENVQCWTSNDNMGFWWILRFPVFLAILINFFIFVRI
VQLLVAKLRARQMHHTDYKFRLAKSTLTLIPLLGVHEVVFAFVTDEHAQGTLRS
AKLFFDLFLSSFQGLLVAVLYCFLNKEVQSELRRRWHRWRLGKVLWEERNTSNH
RASSSPGHGPPSKELQFGRGGGSQDSSAETPLAGGLPRLAESPF

1. A compound comprising:
YX₂QGTX₆TSDX₁₀SIX₁₃LDX₁₆X₁₇AQX₂₀X₂₁FIX₂₄X₂₅LLEGGPSSGEPPPX₃₉,
wherein X₂ is Aib,
X₆ is αMeF(2F),
X₁₀ can be Y or 4Pal,
X₁₃ can be L or αMeL,
X₁₆ is Orn,
X₁₇ is selected from the group consisting of C, D, E, K and Q,
X₂₀ can be 4Pal, Iva or αMeL,
X₂₁ can be A or Aib,
X₂₄ can be E or e,
X₂₅ can be Y or αMeY, and
X₃₉ can be E or S
(SEQ ID NO:5), and
wherein a carboxy-terminal (C-terminal) amino acid optionally is amidated;
or a pharmaceutically acceptable salt thereof.

2. (canceled)

3. The compound of claim 1, wherein X₁₇ is K.

4. The compound of claim 3, wherein the [K] at position X₁₇ and the is conjugated to a C₁₆-C₂₂ fatty acid moiety by a linker between the K at position X₁₇ and the fatty acid moiety.

5. The compound of claim 4, wherein the linker comprises one to four amino acids.

6. The compound of claim 5, wherein the amino acids are E or γE.

7. The compound of claim 4, wherein the linker further comprises the following structure:
H—{NH—CH₂—CH₂—[O—CH₂—CH₂]ₘ—O—(CH₂)ₚ—CO}ₙ—OH,
wherein m is any integer from 1 to 12, n is any integer from 1 to 12, and p is 1 or 2.

8. The compound of claim 4, wherein the linker further comprises one to four (2-[2-(2-amino-ethoxy)-ethoxy]-acetyl) moieties.

9. The compound of claim 1, wherein X₁₇ is K chemically modified through conjugation to an epsilon-amino group of a K side-chain with the following structure:
(2-[2-(2-amino-ethoxy)-ethoxy]-acetyl)ₐ-(γE)_{b}-CO—(CH₂)_{c}—CO₂H,
wherein a is 0, 1 or 2; b is 1 or 2; and c is an integer between 16 to 20.

10. The compound of claim 9, wherein a is 1.

11. The compound of claim 9, wherein a is 2.

12. The compound of claim 9, wherein b is 1.

13. The compound of claim 9, wherein b is 2.

14. The compound of claim 9, wherein c is 20.

15. The compound of claim 1, wherein X₁₀ is Y.

16. The compound of claim 1, wherein X₁₀ is 4Pal.

17. The compound of claim 1, wherein X₁₃ is L.

18. The compound of claim 1, wherein X₁₃ is αMeL.

19. The compound of claim 1, wherein X₂₀ is 4Pal.

20. The compound of claim 1, wherein X₂₀ is Iva.

21. The compound of claim 1, wherein X₂₀ is αMeL.

22. The compound of claim 1, wherein X₂₁ is A.

23. The compound of claim 1, wherein X₂₁ is Aib.

24. The compound of claim 1, wherein X₂₄ is E.

25. The compound of claim 1, wherein X₂₄ is e.

26. The compound of claim 1, wherein X₂₅ is Y.

27. The compound of claim 1, wherein X₂₅ is αMeY.

28. The compound of claim 1, wherein X₃₉ is E.

29. The compound of claim 1, wherein X₃₉ is S.

30. (canceled)

31. A method of treating a disease selected from the group consisting of type 2 diabetes mellitus, dyslipidemia, metabolic syndrome, non-alcoholic fatty liver disease, non-alcoholic steatohepatitis and obesity, the method comprising the step of:
administering to an individual in need thereof an effective amount of a compound claim 1.

32. A method of treating type 2 diabetes mellitus, the method comprising the step of:
administering to an individual in need thereof an effective amount of compound of claim 1.

33. A pharmaceutical composition comprising:
compound of claim 1; and
a pharmaceutically acceptable carrier, diluent, or excipient.

34-43. (canceled)


SEQUENCE LISTING (16 sequences)

SEQ ID NO: 1 - protein, 42 aa, Homo sapiens
YAEGTFISDYSIAMDKIHQQDFVNWLLAQKGKKNDWKHNITQ

SEQ ID NO: 2 - protein, 30 aa, Homo sapiens
  MOD_RES 30: note = AMIDATION
HAEGTFTSDVSSYLEGQAAKEFIAWLVKGR

SEQ ID NO: 3 - protein, 29 aa, Homo sapiens
HSQGTFTSDYSKYLDSRRAQDFVQWLMNT

SEQ ID NO: 4 - protein, 37 aa, Homo sapiens
HSQGTFTSDYSKYLDSRRAQDFVQWLMNTKRNRNNIA

SEQ ID NO: 5 - protein, 39 aa, synthetic construct
  SITE 2: note = X at position 2 is Aib
  SITE 6: note = X at position 6 is (S)-alpha-methyl-2-fluorophenylalanine
  VARIANT 10: note = X at position 10 is Y or 3-(4-Pyridyl)-L-alanine
  VARIANT 13: note = X at position 13 is L or alpha-methyl-L-leucine
  SITE 16: note = X at position 16 is Orn
  VARIANT 17: note = X at position 17 is any amino acid with a functional group available for conjugation
  VARIANT 20: note = X at position 20 is 3-(4-Pyridyl)-L-alanine, isovaline, or alpha-methyl-L-leucine
  VARIANT 21: note = X at position 21 is A or Aib
  VARIANT 24: note = X at position 24 is L-Glutamic acid or D-Glutamic acid
  SITE 25: note = X at position 25 is Y or alpha-methyl-L-tyrosine
  VARIANT 39: note = X at position 39 is E or S
YXQGTXTSDXSIXLDXXAQXXFIXXLLEGGPSSGEPPPX

SEQ ID NO: 6 - protein, 39 aa, synthetic construct
  SITE 2: note = X at position 2 is Aib
  SITE 6: note = X at position 6 is (S)-alpha-methyl-2-fluorophenylalanine
  SITE 16: note = X at position 16 is Orn
  SITE 20: note = X at position 20 is 3-(4-pyridyl)-L-alanine
YXQGTXTSDYSILLDXKAQXAFIEYLLEGGPSSGEPPPE

SEQ ID NO: 7 - protein, 39 aa, synthetic construct
  REGION 1..39: note = Synthetic Construct
  SITE 2: note = X at position 2 is Aib
  SITE 6: note = X at position 6 is (S)-alpha-methyl-2-fluorophenylalanine
  SITE 13: note = X at position 13 is alpha-methyl-leucine
  SITE 16: note = X at position 16 is Orn
  SITE 20: note = X at position 20 is isovaline
  SITE 25: note = X at position 25 is alpha-methyl-L-tyrosine
YXQGTXTSDYSIXLDXKAQXAFIEXLLEGGPSSGEPPPS

SEQ ID NO: 8 - protein, 39 aa, synthetic construct
  SITE 2: note = X at position 2 is Aib
  SITE 6: note = X at position 6 is (S)-alpha-methyl-2-fluorophenylalanine
  SITE 10: note = X at position 10 is 3-(4-pyridyl)-L-alanine
  SITE 13: note = X at position 13 is alpha-methyl-L-leucine
  SITE 16: note = X at position 16 is Orn
  SITE 20: note = X at position 20 is alpha-methyl-L-leucine
  SITE 21: note = X at position 21 is Aib
  SITE 24: note = X at position 24 is D-glutamic acid
  SITE 25: note = X at position 25 is alpha-methyl-L-tyrosine
YXQGTXTSDXSIXLDXKAQXXFIXXLLEGGPSSGEPPPS

SEQ ID NO: 9 - protein, 39 aa, synthetic construct; identical to SEQ ID NO: 6
  SITE 2: note = X at position 2 is Aib
  SITE 6: note = X at position 6 is (S)-alpha-methyl-2-fluorophenylalanine
  SITE 16: note = X at position 16 is Orn
  SITE 17: note = K at position 17 is chemically modified by conjugation of the epsilon-amino group of the K side chain with (2-[2-(2-Amino-ethoxy)-ethoxy]-acetyl)-(gamma-glutamic acid)-CO-(CH2)18-CO2H
  SITE 20: note = X at position 20 is 3-(4-Pyridyl)-L-alanine
  SITE 39: note = Glutamic acid at position 39 is amidated as a C-terminal primary amide

SEQ ID NO: 10 - protein, 39 aa, synthetic construct; identical to SEQ ID NO: 7
  SITE 2: note = X at position 2 is Aib
  SITE 6: note = X at position 6 is (S)-alpha-methyl-2-fluorophenylalanine
  SITE 13: note = X at position 13 is alpha-methyl-L-leucine
  SITE 16: note = X at position 16 is Orn
  SITE 17: note = K at position 17 is chemically modified by conjugation of the epsilon-amino group of the K side chain with (2-[2-(2-Amino-ethoxy)-ethoxy]-acetyl)-(gamma-glutamic acid)-CO-(CH2)18-CO2H
  SITE 20: note = X at position 20 is isovaline
  SITE 25: note = X at position 25 is alpha-methyl-L-tyrosine
  SITE 39: note = Serine at position 39 is amidated as a C-terminal primary amide

SEQ ID NO: 11 - protein, 39 aa, synthetic construct; identical to SEQ ID NO: 8
  SITE 2: note = X at position 2 is Aib
  SITE 6: note = X at position 6 is (S)-alpha-methyl-2-fluorophenylalanine
  SITE 10: note = X at position 10 is 3-(4-Pyridyl)-L-alanine
  SITE 13: note = X at position 13 is alpha-methyl-L-leucine
  SITE 16: note = X at position 16 is Orn
  SITE 17: note = K at position 17 is chemically modified by conjugation of the epsilon-amino group of the K side chain with (2-[2-(2-Amino-ethoxy)-ethoxy]-acetyl)-(gamma-glutamic acid)-CO-(CH2)18-CO2H
  SITE 20: note = X at position 20 is alpha-methyl-L-tyrosine
  SITE 21: note = X at position 21 is Aib
  SITE 24: note = X at position 24 is D-glutamic acid
  SITE 25: note = X at position 25 is alpha-methyl-L-tyrosine
  SITE 39: note = Serine at position 39 is amidated as a C-terminal primary amide

SEQ ID NO: 12 - protein, 10 aa, synthetic construct
GPSSGEPPPE

SEQ ID NO: 13 - protein, 10 aa, synthetic construct
GPSSGEPPPS

SEQ ID NO: 14 - protein, 466 aa, Homo sapiens
MTTSPILQLLLRLSLCGLLLQRAETGSKGQTAGELYQRWERYRRECQETLAAAEPPSGLACNGSFDMYVCWDYAAPNATARASCPWYLPWHHHVAAGFVLRQCGSDGQWGLWRDHTQCENPEKNEAFLDQRLILERLQVMYTVGYSLSLATLLLALLILSLFRRLHCTRNYIHINLFTSFMLRAAAILSRDRLLPRPGPYLGDQALALWNQALAACRTAQIVTQYCVGANYTWLLVEGVYLHSLLVLVGGSEEGHFRYYLLLGWGAPALFVIPWVIVRYLYENTQCWERNEVKAIWWIIRTPILMTILINFLIFIRILGILLSKLRTRQMRCRDYRLRLARSTLTLVPLLGVHEVVFAPVTEEQARGALRFAKLGFEIFLSSFQGFLVSVLYCFINKEVQSEIRRGWHHCRLRRSLGEEQRQLPERAFRALPSGSGPGEVPTSRGLSSGTLPGPGNEASRELESYC

SEQ ID NO: 15 - protein, 463 aa, Homo sapiens
MAGAPGPLRLALLLLGMVGRAGPRPQGATVSLWETVQKWREYRRQCQRSLTEDPPPATDLFCNRTFDEYACWPDGEPGSFVNVSCPWYLPWASSVPQGHVYRFCTAEGLWLQKDNSSLPWRDLSECEESKRGERSSPEEQLLFLYIIYTVGYALSFSALVIASAILLGFRHLHCTRNYIHLNLFASFILRALSVFIKDAALKWMYSTAAQQHQWDGLLSYQDSLSCRLVFLLMQYCVAANYYWLLVEGVYLYTLLAFSVLSEQWIFRLYVSIGWGVPLLFVVPWGIVKYLYEDEGCWTRNSNMNYWLIIRLPILFAIGVNFLIFVRVICIVVSKLKANLMCKTDIKCRLAKSTLTLIPLLGTHEVIFAFVMDEHARGTLRFIKLFTELSFTSFQGLMVAILYCFVNNEVQLEFRKSWERWRLEHLHIQRDSSMKPLKCPTSSLSSGATAGSSMYTATCQASCS

SEQ ID NO: 16 - protein, 476 aa, Homo sapiens
MPPCQPQRPLLLLLLLLACQPQVPSAQVMDFLFEKWKLYGDQCHHNLSLLPPPTLVCNRTFDKYSCWPDTPANTTANISCPWYLPWHHKVQHRFVFKRCGPDGQWVRGPRGQPWRDASQCQMDGEEIEVQKEVAKMYSSFQVMYTVGYSLSLGALLLALAILGGLSKLHCTRNAIHANLFASFVLKASSVLVIDGLLRTRYSQKIGDDLSVSTWLSDGAVAGCRVAAVFMQYGIVANYCWLLVEGLYLHNLLGLATLPERSFFSLYLGIGWGAPMLFVVPWAVVKCLFENVQCWTSNDNMGFWWILRFPVFLAILINFFIFVRIVQLLVAKLRARQMHHTDYKFRLAKSTLTLIPLLGVHEVVFAFVTDEHAQGTLRSAKLFFDLFLSSFQGLLVAVLYCFLNKEVQSELRRRWHRWRLGKVLWEERNTSNHRASSSPGHGPPSKELQFGRGGGSQDSSAETPLAGGLPRLAESPF